(19) United States
(12) Patent Application Publication
(10) Pub. No.: US 2026/0083980 A1
(43) Pub. Date: Mar. 26, 2026

(54) System And Method for Treating the Skin

(71) Applicant: René Shewalter, West Lawn, PA (US)

(72) Inventor: René Shewalter, West Lawn, PA (US)

(21) Appl. No.: 19/336,320

(22) Filed: Sep. 22, 2025

Related U.S. Application Data
(60) Provisional application No. 63/697,636, filed on Sep. 23, 2024.

(51) Int. Cl. A61N 5/067; A61K 9/00; A61K 31/728; A61K 35/12; A61N 5/06

(52) U.S. Cl. CPC A61N 5/067; A61K 9/0014; A61K 31/728; A61K 35/12; A61N 5/0616; A61N 5/0624; A61N 5/0625; A61N 2005/0627; A61N 2005/0663

(57) ABSTRACT

A method for treating a section of skin that includes the epidermis, dermis, and hypodermis. The method includes dermaplaning the section of skin. After dermaplaning, a beam from a non-ablative laser is passed over the section of skin multiple times. The beam of the non-ablative laser further removes dead skin and hair and also warms the underlying dermis and hypodermis. A skin serum is topically applied to the section of skin. The section of skin is treated with electrical arc discharges of a plasma pen to create disruptions in the epidermis. The disruptions created in the epidermis enable the serum to better pass into and through the epidermis. The treated area of skin can then be subjected to light therapy to increase blood flow, promote the healing of the created disruptions, and provide protection against infection.

### RELATED APPLICATIONS

This application claims the benefit of U.S. Provisional Patent Application No. 63/697,636, filed Sep. 23, 2024.

### BACKGROUND OF THE INVENTION

### 1. Field of the Invention

In general, the present invention relates to systems and methods for treating the skin for the purpose of diminishing areas of darker pigmentation and other aesthetic skin anomalies typically associated with aging. More particularly, the present invention relates to skin treatment methods that use exfoliation, laser treatments, and plasma probe treatments in combination.

### 2. Prior Art Description

As a person ages, it is not unusual for the skin to lose elasticity and to develop areas of uneven pigmentation, such as age spots. There are countless creams, serums, and other topically applied products that are marketed to treat aging skin. Likewise, plastic surgeons and dermatologists have many medical techniques that are used to make the skin appear more youthful. However, most techniques involve the use of only a single treatment type, such as dermabrasion or laser treatment. Few techniques use multiple treatment types, wherein each treatment type is intended to prepare the skin to make the skin more receptive to subsequent treatments.

There are some instances of procedures that include more than one treatment type. However, the treatments are layered and are not intended to be synergistic. That is, each procedure does not make the prior procedure or subsequent procedures any more or less effective. For example, in U.S. Pat. No. 8,945,104, to Boone, a microabrasion device is disclosed that also has the ability to shine light. As such, one device applied to the skin can create microabrasions in the skin as the skin is simultaneously exposed to light. The therapy provided by the microabrasions is not improved by the application of light and vice versa. The system merely combines the need for two tools into one.

U.S. Patent Application No. 2007/0156124 to Ignon describes a system where a microabrasion head is combined with a fluid dispenser. In this manner, fluid can be applied to the skin during the microabrasion procedure. Although the presence of fluid may help with skin friction, here too, the system merely combines the need for two tools into one.

The present invention represents an advancement in skin treatment methodologies, wherein multiple treatment techniques are used in a synergistic manner that makes each step in the methodology more effective. The result is a treatment to the skin that is more effective as a whole than would be possible if the components were employed alone. The improved skin treatment methodology is described and claimed below.

### SUMMARY OF THE INVENTION

The present invention is a method for treating a section of skin that includes the epidermis, dermis, and hypodermis. The method includes dermaplaning the section of skin to shave away dead skin and hair from the section of skin. After dermaplaning, a beam from a non-ablative laser is passed over the section of skin multiple times. The beam of the non-ablative laser further warms the underlying dermis and hypodermis.

A skin serum is topically applied to the section of skin. The section of skin is treated with electrical arc discharges of a plasma pen to create disruptions in the epidermis. The disruptions created in the epidermis enable the serum to better pass into and through the epidermis. The treated area of skin can then be subjected to light therapy to increase blood flow, promote the healing of the created disruptions, and provide protection against infection.

### BRIEF DESCRIPTION OF THE DRAWINGS

For a better understanding of the present invention, reference is made to the following description of an exemplary embodiment thereof, considered in conjunction with the accompanying drawings, in which:

FIG. 1 shows a segment of skin undergoing a dermaplaning procedure in a first step of a skin treatment procedure;

FIG. 2 shows a segment of skin undergoing a laser procedure using a non-ablative laser in a second step of a skin treatment procedure;

FIG. 3 shows a segment of skin undergoing a serum application procedure in a third step of a skin treatment procedure;

FIG. 4 shows a segment of skin undergoing a plasma probe procedure in a fourth step of a skin treatment procedure; and

FIG. 5 shows a segment of skin undergoing a light exposure procedure in a fifth step of a skin treatment procedure.

### DETAILED DESCRIPTION OF THE DRAWINGS

Although the present invention system and methodology can be embodied in many ways, only one exemplary embodiment is illustrated and described. The exemplary embodiment is being shown for the purposes of explanation and description. The exemplary embodiment is selected in order to set forth one of the best modes contemplated for the invention. The illustrated embodiment, however, is merely exemplary and should not be considered a limitation when interpreting the scope of the appended claims.

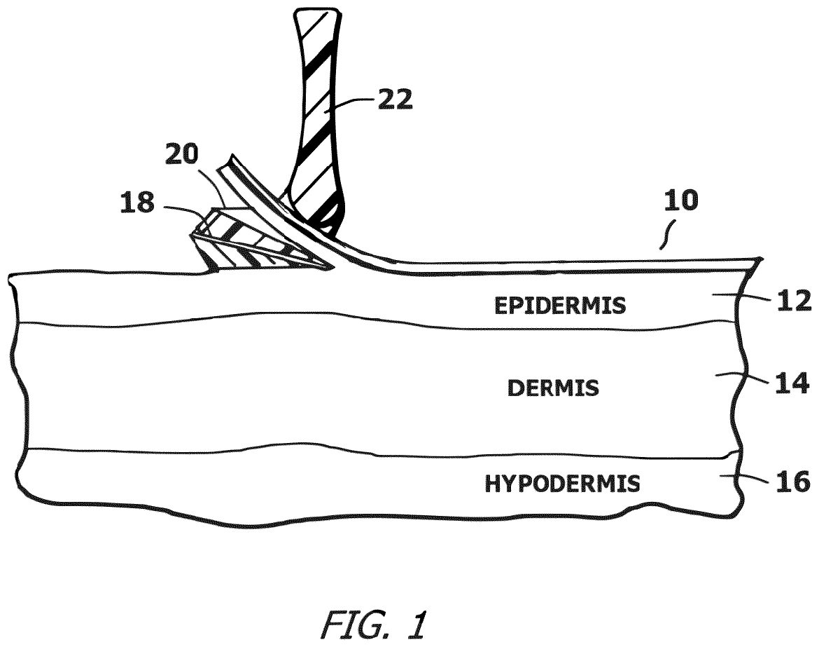

Referring to FIG. 1, a section of skin 10 is shown having an epidermis 12, a dermis 14 and a hypodermis 16. The section of skin 10 should be considered as an example of a segment of skin that contains a pigment discoloration, scar tissue, wrinkling, or some other aesthetic issue of the skin. The present invention methodology treats the segment of skin 10 in a multi-step process. Each step of the process makes the segment of skin 10 better prepared for the subsequent step(s). As such, the process steps in totality act synergistically to improve the health of the segment of skin 10.

The first process step is shown in FIG. 1. In FIG. 1, the section of skin 10 is first treated with a dermaplaning process. During the dermaplaning process, some of the epidermis 12 is mechanically shaved away using a blade 18. The blade 18 is commonly referred to as an exfoliation scalpel or a drum dermatome. The blade 18 is gently shaved across the section of skin 10 to remove the top layer of dead skin cells and surface hair. This process helps the section of skin 10 to absorb products better and to improve the texture of the skin. Accordingly, the first step helps the section of skin 10 to absorb serums that may be later applied. The process also boosts the production of collagen, which improves the texture and tightness of the section of skin 10.

The blade 18 is preferably a 10R butter blade scalpel head 20 mounted to a handle 22. However, other scalpels, including electric scalpels and scrapers, can also be used. Abrasion pads can also be used. What is of importance is that most dead skin and hair is mechanically removed from the section of skin 10.

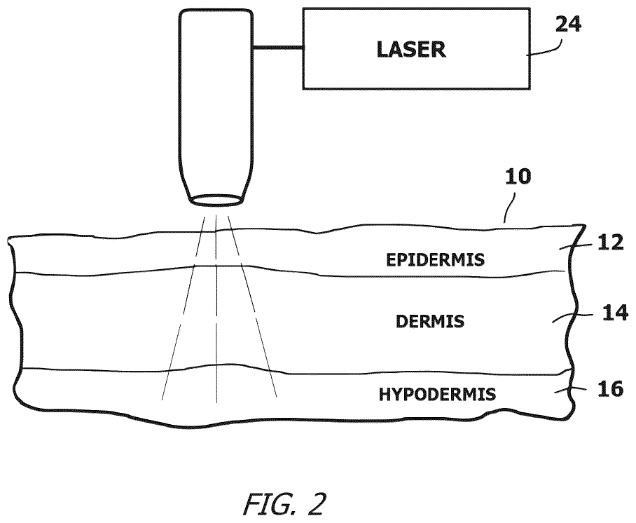

In FIG. 2, the second step of the methodology is shown. Referring to FIG. 2 in conjunction with FIG. 1, it can be seen that once the skin 10 is conditioned with the initial dermaplaning process, the skin 10 is then subjected to a laser treatment with a non-ablative laser 24. The non-ablative laser 24 heats the softened skin left behind after the dermaplaning procedure. The non-ablative laser 24 directly shines upon the epidermis 12, but passively heats the underlying layers of the dermis 14 and hypodermis 16. This stimulates the growth of collagen, therein improving the firmness and texture of the section of skin 10. As the area of skin 10 heals and skin regrows after the laser exposure, the treated area of skin 10 is smoother and tighter. The non-ablative laser 24 can be a carbon dioxide (CO2) laser, an erbium laser and combination laser systems. However, the preferred non-ablative laser 24 is an Nd:YAG pulsed laser having a wavelength at, or near, 1064 nm and with a fluence range of up to 318 Joules per square centimeter. Such a laser is the Aerolase® NEO Elite laser as described in U.S. Pat. No. 7,298,763 to Grunzdev.

The non-ablative laser 24 is used to make three sets of passes over the section of skin 10. The non-ablative laser 24 is set at different power levels for each set of passes. The non-ablative laser 24 is initially set at a first power level for a plurality of passes over the area of skin 10. The first power level is at or near the half power setting of the preferred laser previously described. The number of passes depends upon the thickness of the area of skin 10 and the depth of the aesthetic defect being treated. Typically, between two and ten passes of the non-ablative laser 24 are used. After a first set of laser passes is complete, the non-ablative laser 24 is then set to a higher power level for a second set of passes. Since the non-ablative laser 24 is now at a higher power setting only a few (2-8) passes are typically required. After the high-power set of passes is complete, a third set of laser passes are made. In the third set of passes, the power of the non-ablative laser 24 is set to a medium power level that is lower than the second set of passes, but higher than the first set of passes. Counting all three sets of passes, the number of laser passes in total is preferably between twelve and twenty-five.

The non-ablative laser treatment diminishes pigment abnormalities and reduces the appearance of pores, rhytids, and wrinkles on the epidermis 12. The laser treatment heats the underlying dermis 14 and the hypodermis 16. This stimulates the capillaries to increase blood flow, while simultaneously promoting the production of collagen to improve the elasticity of the section of skin 10.

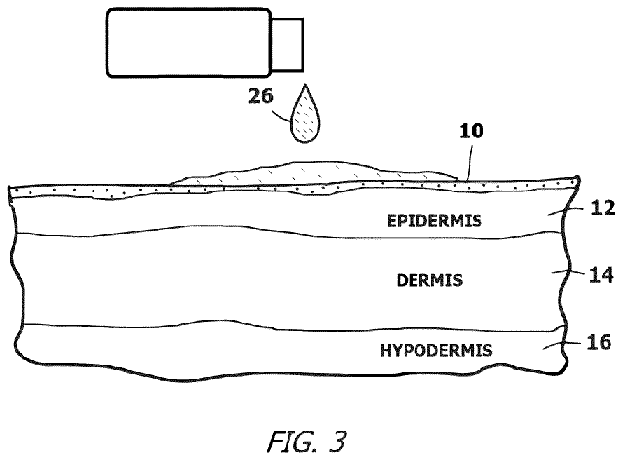

In FIG. 3, the third step of the methodology is shown. Referring to FIG. 3, it can be seen that once the skin 10 is shaved with the dermaplaning procedure of FIG. 1 and conditioned with the non-ablative laser procedure of FIG. 2, the section of skin 10 is treated with a humectant serum 26, such as a hyaluronic acid serum or an exosome serum. The humectant serum 26 absorbs water to hydrate and plump the section of skin 10 by attracting and retaining moisture within the skin. By retaining moisture, humectant serum 26 helps to make the section of skin 10 look fuller and more supple. This helps to reduce the appearance of fine lines and wrinkles and improves the elasticity and texture of the section of skin 10. The humectant serum 26 fortifies the skin's natural barrier to protect it from dryness, irritation, and environmental damage. The humectant serum 26 is better absorbed by the skin 10 due to the previous dermaplaning and laser treatments that reduce and soften the epidermis 12.

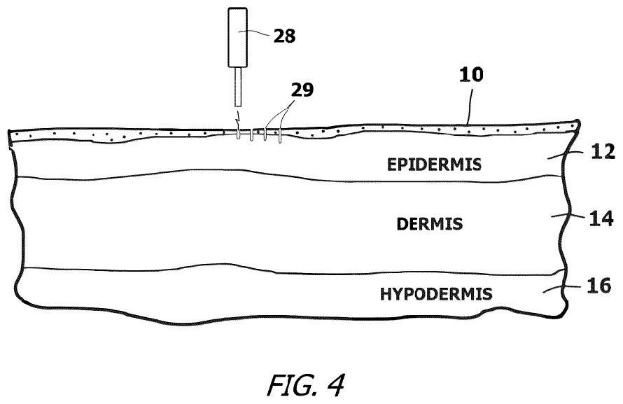

In FIG. 4, the fourth step of the methodology is shown. Referring to FIG. 4 in conjunction with FIG. 3, it can be seen that with the humectant serum 26 applied to the treated section of skin 10, the humectant serum 26 is driven deeper into the skin 10 using a plasma pen 28. The plasma pen 28 is a lightweight hand-held plasma device that delivers controlled discharges to the section of skin 10 that disrupts the thinned epidermis 12. The plasma pen 28 generates an electrical arc from the tip of the plasma pen 28 to the section of skin 10, ionizing the atmospheric gas to create a small plasma field. The plasma energy creates pinpoint arcs of plasma that cause tiny disruptions 29 on the section of skin 10. These skin disruptions 29 stimulate fibroblast and new collagen formation which results in a tightening of the tissue. Furthermore, the plasma pen 28 disrupts the epidermis 12 and enables more of the humectant serum 26 to reach and affect the underlying dermis 14. Accordingly, the plasma pen 28 works synergistically with the humectant serum 26 creating disruptions in the epidermis 12 that enable the humectant serum 26 to better penetrate the epidermis 12 and reach the underlying dermis 14.

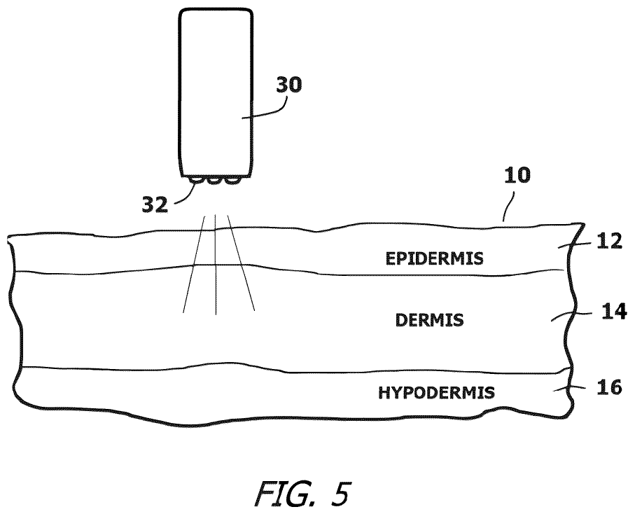

Referring to FIG. 5, an optional fifth method step is disclosed. In the fifth step, the section of skin 10 is subjected to light therapy while the humectant serum 26 is still being absorbed by the section of skin 10. The section of skin 10 is exposed to a light fixture 30 that contains LEDs 32 of different wavelengths. The section of skin 10 is first subjected to red light having a preferred primary wavelength of 625+/−3 nm. The red light keeps the section of skin 10 warm and increases blood flow so that the humectant serum 26 can be better absorbed.

After the application of red light, the red light is changed to, or mixed with, blue light near 415 nm. The blue light kills bacterium on and within the section of skin 10 while diminishing inflammation. This helps prevent infection and acne breakouts in the treated section of skin 10. Other wavelengths of light can be used for other purposes. For example, green wavelengths of light can be used to decrease pain on the section of skin 10 if the skin is sensitive after the prior skin treatments.

Referring to all figures, it will now be understood that the overall effect of all described steps is synergistic. The dermaplaning process of FIG. 1 cleans and thins the epidermis 12. The non-ablative laser 24 of FIG. 2 evens skin pigmentation and revitalizes the underlying dermis 14 and hypodermis 16. The humectant serum 26 as applied in FIG. 3 helps the area of skin 10 to retain moisture. The use of the plasma pen 28, as shown in FIG. 4, helps the humectant serum 26 penetrate the epidermis 12 to reach the dermis 14 while adding its own beneficial effects to the dermis 14. Lastly, the light therapy of FIG. 5 helps the humectant serum 26 be absorbed by the section of skin 10 while inhibiting any infections from developing. The result is an overall methodology that is highly beneficial to the section of skin 10.

It will be understood that the methodology of the present invention that is illustrated and described is merely exemplary and that a person skilled in the art can make many variations to the shown embodiment. All such embodiments are intended to be included within the scope of the present invention as defined by the claims.

What is claimed is:

1. A method for treating a section of skin that includes the epidermis, dermis and hypodermis, said method comprising:
dermaplaning said section of skin to remove dead skin and hair from said section of skin;
passing a beam from a non-ablative laser over said section of skin to further remove dead skin and hair and to warm said dermis and said hypodermis;
topically applying a serum to said section of skin; and
treating said section of skin with a plasma pen to create disruptions in said epidermis to enable said serum to better pass into and through said epidermis.

2. The method according to claim 1, further including treating said section of skin with red light to increase blood flow within said section of skin and induce healing of said disruptions.

3. The method according to claim 1, further including treating said section of skin with blue light to inhibit bacterial growth on said section of skin.

4. The method according to claim 1, wherein dermaplaning said section of skin includes shaving said section of skin with a blade to remove said dead skin and said hair.

5. The method according to claim 1, wherein passing a beam from a non-ablative laser includes making a first set of passes with said beam over said section of skin with said non-ablative laser at a first power setting.

6. The method according to claim 5, wherein passing a beam from a non-ablative laser includes making a second set of passes with said beam over said section of skin with said non-ablative laser at a second power setting that is higher than said first power setting.

7. The method according to claim 6, wherein passing a beam from a non-ablative laser includes making a third set of passes with said beam over said section of skin with said non-ablative laser at a third power setting that is between said first power setting and said second power setting.

8. The method according to claim 7, wherein said serum is a humectant serum selected from a group comprised of hyaluronic acid serums and exosome serums.

9. The method according to claim 2, wherein said red light has a median wavelength of 625 nm +/−3 nm.

10. The method according to claim 3, wherein said blue light has a median wavelength of 415 nm.

11. The method according to claim 1, wherein passing a beam from a non-ablative laser over said section of skin includes passing said beam over said section of skin between twelve and twenty-five times.

12. A method for treating a section of skin that includes the epidermis, dermis and hypodermis, said method comprising:
shaving said section of skin with a blade to remove dead skin and hair from said section of skin;
lasering said section of skin to warm said dermis and said hypodermis;
topically applying a humectant to said section of skin; and
creating disruptions in said epidermis to enable said serum to better pass into and through said epidermis.

13. The method according to claim 12, wherein creating disruptions in said epidermis includes creating disruptions with electrical arc discharges.

14. The method according to claim 12, further including treating said section of skin with red light to increase blood flow within said section of skin and induce healing of said disruptions.

15. The method according to claim 12, further including treating said section of skin with blue light to inhibit bacterial growth on said section of skin.

16. The method according to claim 12, wherein lasering said section of skin includes making a first set of passes with a beam from a non-ablative laser over said section of skin with said non-ablative laser at a first power setting.

17. The method according to claim 16, wherein lasering said section of skin includes making a second set of passes with said beam at a second power setting that is higher than said first power setting.

18. The method according to claim 17, wherein lasering said section of skin includes making a third set of passes with said beam at a third power setting that is between said first power setting and said second power setting.

19. The method according to claim 12, wherein said humectant is a skin serum selected from a group comprised of hyaluronic acid serums and exosome serums.
